(19) United States
(12) Patent Application Publication
(10) Pub. No.: US 2018/0008735 A1
(43) Pub. Date: Jan. 11, 2018

(54) SANITIZING APPARATUS SUPPORT

(71) Applicant: Stephen ALMEIDA, Tampa, FL (US)

(72) Inventor: Stephen ALMEIDA, Tampa, FL (US)

(73) Assignee: SanitiZall Corp., Scarborough, ME (US)

(21) Appl. No.: 15/202,684

(22) Filed: Jul. 6, 2016

(51) Int. Cl. A61L 2/10

(52) U.S. Cl. CPC A61L 2/10; A61L 2202/11

(57) ABSTRACT

The present disclosure presents sanitization devices and methods. More particularly, the disclosure presents devices and methods that significantly reduce or eliminate the activity of germs, bacteria and/or other infectious microorganisms from a variety of objects. The disclosure describes a flexible support for holding and suspending objects in the sanitizing device which improves the level and intensity of the sanitization process.

### FIELD OF DISCLOSURE

The present disclosure relates to sanitization devices and methods. More particularly, the disclosure relates to devices and methods that significantly reduce or eliminate the activity of germs, bacteria and/or other infectious microorganisms from a variety of objects. The disclosure describes a flexible support for holding and suspending objects in the sanitizing device which improves the level and intensity of the sanitization process.

### BACKGROUND OF THE DISCLOSURE

The surfaces of many objects used in everyday life tend to attract and harbor potentially harmful infectious organisms, such as microbes, pathogens, viruses, bacteria and the like. Particularly infected are objects that are passed from one person to another without the benefit of cleaning or sanitizing. Objects that are handled or breathed-on by different people, or come in contact with surfaces contaminated by other people or animals, can themselves become contaminated. If these objects then contact another person, they can transmit diseases. Even the hands and clothing of medical or healthcare personnel can serve to transmit diseases. This contamination problem is particularly acute with objects used continually by different people. If they are not sterilized between the different users of the objects they can serve as the vector to transmit the disease from one person to the next. Examples include TV remotes in hotel rooms and medical devices. Also objects that come into contact with children are often contaminated with germs that may be infectious if the objects are not routinely cleaned and may harbor media in which infectious germs may breed over time. Sports equipment is another class of objects that over time may become breeding ground for potentially harmful organisms. Objects as large as furniture often become contaminated on the surface with pathogens which are very difficult to remove.

One method of reducing the impact of infectious organisms is to expose them to germicidal ultraviolet bulbs. The bulbs are generally short wave low pressure mercury vapor tubes that produces ultraviolet wavelengths that are lethal to micro-organisms. Approximately 95% of the ultraviolet energy emitted by these bulbs is at and around the mercury resonance line of 254 nanometers. This wavelength is in the region of maximum germicidal effectiveness and is highly lethal to virus, bacteria and mold spores. It deactivates the DNA of bacteria, viruses and other pathogens and thus destroys their ability to multiply and cause disease. Specifically, UV-C light causes damage to the nucleic acid of microorganisms by making them form covalent bonds between certain adjacent bases in the DNA. The formation of such bonds prevents the DNA from being unzipped for replication, and the organism is unable to reproduce. In fact, when the organism tries to replicate, it dies. UVC radiation has extremely low penetrating ability and does not penetrate past the dead-cell layers of the skin. UV will cause eye irritations or burns after prolonged exposure. Recently UVC-radiating LED light have become available and are useful as germicidal devices.

Devices for sanitizing objects that might have harmful infectious organisms on their surfaces have been described, for example, U.S. Pat. Application 2005/0063922A1 to Wesley et al. The devices use sanitizing bulbs that emit sanitizing radiation and shine that radiation onto the surfaces of the object to be sanitized. The sanitizing radiation is a light-of-sight process in which the radiation impinges directly onto the surfaces which are in an unimpeded straight line from the source to the surface. As such, anything in the way of the direct line from the irradiation source to the surface, such as dirt particles and the like on the surface, will prevent those covered surface areas from being sanitized. This is a recognized problem with the devices currently available. One method to improve the problem, in one case, is by rotating the object to be sanitized so that more surface can be aligned in a line-of-line configuration with the radiation source as the device rotates. Another method to improve this problem is by providing mirrors and/or mirrored surfaces that reflect the sanitizing radiation at different angles to the surface areas to be sanitized.

In order for an object to be sanitized in a sanitizing device, the object must be placed in a holder, a cradle or lie on a support. The holder, cradle or support then covers an area of the object with which that it comes into contact, thereby preventing the sanitizing radiation from impinging onto that area and sanitizing it. Holder, cradles and supports may be made from materials which allow some sanitizing radiation to pass through. For example, in US Pat. APP. 2014/0264075 to LaPorte et al, a support for a personal electronic device is placed on a flat support made of “transparent” materials such as glass, plastic, polymer, ceramic or other suitable materials. While it is known that a small amount of UVC might pass through such materials, it is also well known that the intensity of the sanitizing radiation must be increased in order to allow the amount required to kill pathogen organism to pass through. Since the absorbance of the materials that make the holder, cradle, or support is related to the thickness of the holder, cradle or support, (Beer's Law) the amount of radiation intensity needs to exponentially increase with thickness. One side effect of increasing intensity of the sanitizing lamps is the potential damage to the object and/or the device as sanitizing radiation is very energetic; the less radiation needed the better for the shelf life and stability of the materials that make up the object as well as the device itself.

Thus there is an unmet need to improve sanitizing devices such that objects to be sanitized can be held in the device in an effective and efficient manner to allow sanitizing radiation to sanitize objects without having to grossly intensify the radiation output from the sanitizing lamps.

### SUMMARY OF THE EXEMPLARY EMBODIMENTS

It is an object of the current disclosure to overcome the deficiencies commonly associated with the prior art as discussed above and provide devices and methods that improve the elimination or significant reduction of the activity of infectious microorganisms from objects. The present disclosure relates to sanitization devices and methods. More particularly, the disclosure relates to devices and methods that significantly reduce or eliminate the activity of germs, bacteria and/or other infectious microorganisms from a variety of objects. The disclosure describes a support for holding and suspending objects in the sanitizing device which improves the level and intensity of the sanitization process. Objects for sanitization range from hand-held devices, such as TV remotes, cell phones, electronic devices and personal and other handheld devices, to larger objects such as sports equipment, objects used for infant care, and the like. The device and method uses a flexible support component into which an object is placed for sanitization. The flexible support allows the object to be suspended in the sanitizing device such that all parts of the device are exposed to the radiation. The support is a thin film, a mesh, a woven fabric or a perforated film. The support is made of materials which allows sanitizing radiation to pass through and directly impinge on the object for sanitization. The support is made from materials that are flexible and conformable and allow substantial transparency to sanitizing radiation, to allow germicidal radiation to exposes areas of the object that would not be exposed if the object were supported by a solid support.

In a first embodiment, disclosed and claimed herein is an apparatus for sanitizing an object including a compartment having a top portion, a bottom portion and side portions and configured to envelop the object to be sanitized, at least one radiation source within the compartment, wherein the at least one radiation source can provide UV radiation capable of substantially deactivating infectious organisms, and a flexible support for holding and suspending the object to be sanitized within the compartment. The flexible support comprises material capable of transmitting radiation capable of substantially deactivating infectious organisms.

In a second embodiment, disclosed and claimed herein is an apparatus for sanitizing an object of the above embodiment wherein the support comprises glass, a polymer or a combination.

In a third embodiment, disclosed and claimed herein are apparatuses for sanitizing an object of the above embodiments, wherein the support comprises polymethylmethacrylate, fluoropolymers, fluorinated poly-co-ethylene-propylene, polythene, silicone, polymethylpentene, polyolefin, fluorinated polyolefin, cyclic olefin polymers, fluorinated cyclic polyolefin, polyethylene, perfluorinated polyethylene, polypropylene, perfluorinated polypropylene, or combination thereof.

In a fourth embodiment, disclosed and claimed herein are apparatuses for sanitizing an object of the above embodiments, wherein the support is a thin film, perforated sheet, a woven construction or a mesh is flexible and conformable around at least a portion of the object.

In a fifth embodiment, disclosed and claimed herein are apparatuses for sanitizing an object of the above embodiments, further comprising a rotating mechanism for rotating the support.

In a sixth embodiment, disclosed and claimed herein are apparatuses for sanitizing an object of the above embodiments, wherein the UV radiation is about 250 to about 280 nm as well as about 253.7 nm

In a seventh embodiment, disclosed and claimed herein are apparatuses for sanitizing an object of the above embodiments wherein the apparatuses further comprise a component for the removal of debris from the surface of the object to be sanitized.

In an eighth embodiment, disclosed and claimed herein are apparatuses for sanitizing an object of the above embodiments further comprising electronic connectors for charging devices, connecting electronic devices, communication devices, internet devices, and combinations thereof.

In a ninth embodiment, disclosed and claimed herein are apparatuses for sanitizing an object of the above embodiments wherein the flexible support is removable from the compartment.

In further embodiments, disclosed and claimed herein are apparatuses for sanitizing an object of the above embodiments wherein the compartment is sized to accept cell phones and TV remotes, other handheld devices, smart pads, keypads, jewelry, keys, credit cards, money, toiletry items, food related items including food items that come into contact with food, such as trays, dental and medical equipment and devices, writing utensils, kitchen utensils, books, magazines, gaming items like dice and playing cards, toys, balls, personal hygiene items, such as combs, brushes, toothbrushes, baby related objects like rubber nipples, containers, cordless telephone, smart phones, wireless headsets, portable media devices, digital cameras, video recorders, audio recorders, portable gaming devices, portable computing devices, tablet computers, laptop computers, notebook computers, electronic reading devices, personal digital assistants (PDA), palmtop computers, handheld computers, pen computers, ultra-mobile personal computers, pagers, portable navigation devices, personal navigation assistants, for example, portable Global Positioning System units, personal electronic devices, toys, balls, personal hygiene devices, devices used by infants, containers, portable data devices, gaming items, toiletry items, food items, dental and medical instruments, writing utensils, kitchen utensils, books, magazines, and other objects which can carry infectious organisms. Also included are larger objects such as furniture and televisions, computers, printers, and the like.

In further embodiments, disclosed and claimed herein are methods of sanitizing an object, including the steps of providing one of the apparatuses of the above embodiments, positioning the object to be sanitized in the flexible support, irradiating the object with radiation from the one or more radiation source, and removing the object from the apparatus.

### BRIEF DESCRIPTION OF THE FIGURES

FIG. 1 is a photo of one embodiment of the flexible support of the current disclosure

FIG. 2 is a graphic depiction of one embodiment of the flexible support of the current disclosure

### DETAILED DESCRIPTION OF THE DISCLOSURE

The term “compartment” as used herein means the enclosure into which a device for sanitization is placed and wherein sanitization is to take place. The compartment may be configured to enclose small hand-held devices as well as larger devices, such as a television, furniture, computers, sports equipment and the like.

The term “apparatus” is used herein to include the compartment and any other peripheral components.

As used herein the term “envelope” means to completely surround and enclose.

As used herein the term sanitized refers to a reduction of the activity of infection organisms and is not meant to infer 100% elimination of all infectious organisms.

As used herein the term “deactivate” means that the infectious organism is rendered non-infectious, which includes the killing of the organisms.

As used herein the term “UV radiation” refers to any UV radiation that is capable of deactivating pathogen organisms, for example UVC and UVB radiation.

As used herein the term “transparent” refers to the property of materials which allow at least a portion of desired UV radiation to pass through.

Disclosed and claimed herein is an apparatus for sanitizing an object, such as, for example, the surface of an object, including a compartment having a top portion, a bottom portion and side portions and configured to envelop the object to be sanitized, at least one radiation source within the compartment, wherein the at least one radiation source can provide UV radiation for substantially deactivating infectious organisms and a flexible support for holding and suspending the object to be sanitized within the compartment. The flexible support comprises material capable of transmitting radiation capable of substantially deactivating infectious organisms.

The apparatus may be constructed from any of a number suitable materials, such as, for example, lightweight, rigid plastic, plastic composite, thermoplastic, radiation cured materials, metal, or a combination. Suitable plastics include polyvinyl chloride, polypropylene, polyolefin, acrylonitrile-butadiene-styrene, polyethylene, polyurethane, polycarbonate, high-impact polystyrene, nylon or blends thereof. The apparatus may be constructed so that the compartment completely encloses an object to be sanitized when in use. Likewise the compartment may be constructed so that only a portion of the object to be sanitized is enclosed thereby allowing partial sanitization of the object as desired. The apparatus and compartment may take on any of a number of shapes or styles chosen for its utility, style, or eye-appeal. For example the apparatus and compartment may be higher than it is wide or vice versa. The sidewalls could be vertical or could be slanted in or out depending on the desired design of the device. The overall shape may be square, rectangular, triangular, oval, cylindrical, essentially spherical with a flat stabilizing bottom, or other appealing polygonal shape. The apparatus may also include a stabilizing base such that the apparatus is stabilized to falling over.

An ingress may be positioned anywhere desirable in the compartment of the apparatus depending on the method used for placing the object to be sanitized into the compartment, such as through the top, the sides, the bottom or a combination. The ingress is configured to allow the object to be sanitized to be placed into the flexible support in the compartment. A mechanism to enclose the compartment is also present which, in operation, will cover the ingress and not allow any substantial amount of sanitizing radiation to be emitted. This can be a hinged cover, a removable cover, a cap, a sliding cover, or other mechanism known in the art to cover an opening.

The ingress may be designed so that the object to be sanitized may be placed by hand into the flexible support in the compartment of the apparatus. This may be through the top, the side or a combination of both.

In another embodiment, the ingress may result from a “clam-shell” type opening in which the top and a portion of all sides of the apparatus are capable of being opened to allow the object to be sanitized to be placed into the flexible support in the compartment and the clam-shell closed over the object.

The compartment contains one or more one or more bulbs which emit radiation that deactivates infectious organisms. The radiation includes, for example, UV radiation, UVC radiation and other wavelengths that deactivate infectious organisms, including radiation emitted at a wavelength around 254 nm. The current disclosure is not limited to only bulbs, but includes other devices which emit sanitizing radiation, such as, for example LED devices.

The most effective wavelength for killing or inactivating microorganisms is the 100-290 nm range, which is the UVC wavelength band. It is composed of short wavelengths from 200 to 280 nm. Most commercially available UVC bulbs are low pressure mercury vapor bulbs that give off a wavelength of 254 nm, which is near the optimum for killing or inactivating microorganisms. Low-pressure mercury-vapor bulbs usually are made with a quartz bulb in order to allow the transmission of short wavelength light. Natural quartz allows the 254 nm wavelength to pass through but blocks the 184 nm wavelength. Synthetic quartz may also be used which allows the 184 nm wavelength to pass, however 184 nm can produce ozone. The bulbs are generally doped with materials that suppress or eliminate the 184 nm wavelengths in low-pressure mercury vapor bulbs.

Light emitting diodes (LED) useful for the current disclosure emit between about 250 nm to about 280 nm, which as mentioned above is well within the germicidal range.

Not to be held to theory, a wavelength between about 250 nm to about 280 nm, such as for example, 253.8 nm UV will break down the molecular bonds within the DNA of micro-organisms producing thymine dimers in their DNA thereby destroying them, rendering them harmless or prohibiting growth and reproduction. It is a process similar to the UV effect of longer wavelengths UVB on humans. However UVB and UVA do not act as sanitizing radiations.

As an example, commercially available T5 size UVC germicidal bulbs range in input power from about 7-16 watts for a tube which is 11.3 inches long. Output wattage for these bulbs, consisting primarily of 254 nm emissions, is approximately 2-4 watts with an efficiency rating of between about 20 and about 40 μW/cm² at a distance of 1 meter from the tube. Power intensity of approximately 1400 to 2800 μW/cm² measured at a distance of 2 inches from the bulb surface is achieved. Alternatively, commercially available LEDs may be used which emit between about 250 nm and about 280 nm. Again not to be held to theory, it has been reported that to reach a 99% kill rate of bacillus anthracis a dosage of 8,700 μW second/cm² is required. Thus, in the current example and using the equation: Intensity×Exposure Time=μW second/cm², a bulb with a minimum power intensity of 1400 μ/cm² at 2 inches from the bulb surface, an exposure time of less than 7 seconds is required. Of course a longer time will improve the kill rate for bacillus anthracis. Other notable 99% kill rate exposure requirements for UVC (measured in μW/cm²) are: E. coli=6500, Salmonella typhosa=6000, Dysentery=4200 and Cholera=6500. It should be noted that in the example a 7 second exposure would be sufficient to provide a 99% kill rate of all the aforementioned bacteria. Viruses are also killed by UVC, some of the toughest being poliovirus and rotavirus, which require 21,000 μW/cm² for a 99% kill rate. Thus using the bulbs of the above example, a 15 second exposure would provide a 99% kill rate. Also molds and yeasts can be killed by UVC exposure. Other UV wavelengths are known to deactivate certain infectious organisms and are included here in the current disclosure.

Although UVC radiation is the most efficient and effective radiation for deactivating microorganisms, other UV radiation is also useful for various microorganism such as UVB radiation.

Sanitizing bulbs may be chosen for their specific radiation output. For example, bulbs may put out both about 184 nm light as well as light around 254 nm. In this case there may be an amount of ozone generated, which can be helpful in sanitization, but can be considered toxic. Or the bulbs can be chosen to only put out about 254 nm radiation. If there is ozone generated by the bulbs, an ozone absorbing material may be present as a part of the compartment or the apparatus which substantially absorbs the ozone to a safe level before it can be released into the general atmosphere.

The apparatus contains a flexible support positioned inside the compartment into which is place all or a portion of the object to be sanitized. The flexible support is constructed from material that is capable of transmitting radiation capable of substantially deactivating infectious organisms.

Not to be held to theory, it should be pointed out that the amount of radiation that passes through a material is controlled by the Beer-Lambert Law, which by definition, the transmittance of a material sample is related to its optical depth τ and to its absorbance A as

$$T = \frac{{Φ}_{e}^{t}}{{Φ}_{e}^{i}} = {e}^{- r} = {10}^{- A} ,$$

### Where

Φₑ^{t} is the radiant flux transmitted by that material sample;
Φₑⁱ is the radiant flux received by that material sample.

As can be seen if a material has very low absorbance A, the transmission will be very high. As well if a material has a small optical depth τ, the transmission will be very high.

Optical depth, or optical thickness, is the natural logarithm of the ratio of incident to transmitted radiant power through a material. It is dimensionless and is a monotonically increasing function of the path length of the radiant power and approaches zero as the path length approaches zero.

Thus, the methods of increasing the transmittance of radiation, in this case, UVC and UVB, through a material are to have a material with low absorbance of UVC and/or UVB, or to decrease the path length through which the radiation needs to pass, such as, providing thin materials, such as threads.

Materials useful for the current disclosure include glass, polymers or a combination thereof. Useful polymers that are known to allow UVC and/or UVB to be transmitted, include, for example, polymethylmethacrylate, fluoropolymers, fluorinated poly-co-ethylene-propylene, polythene, silicone, polymethylpentene, polyolefin, fluorinated polyolefin, cyclic olefin polymers, fluorinated cyclic polyolefin, polyethylene, perfluorinated polyethylene, polypropylene, perfluorinated polypropylene, or combination thereof.

These materials can be made into fibers and thus fabrics, meshes and sheets with perforations. In this way there is less material through which the sanitizing radiation is requires to transmit.

The flexible support made from fibers, may be, for example, a woven fabric. The fabric may be loosely woven which allows for much open space between the threads to allow a large amount of sanitizing radiation to impinge the object to be sanitized without being transmitted through a material. The thickness of the threads is chosen to be sufficiently strong enough to hold the desired objects. For example, sanitizing a bowling ball would require thicker fibers for the support than sanitizing a cell phone. The support may also be a mesh in which fibers that make up the support are connected at the point where the fibers cross, though such methods as melting the fibers, if they are made of plastic.

The flexible support may also be a perforated sheet, with an array of perforations which has enough structure to hold and support the object to be sanitized, again reducing the amount of material required through which the sanitizing radiation is required to transmit. Larger, heavier objects will of course need to have a support with more structure, which can be obtained by having less perforations or having thicker, and consequently stronger non-perforated material.

As can be seen from the foregoing discussion, the flexible support using threads, meshes, fabrics and perforated sheets is primarily open so that a major portion of sanitizing radiation may impinge on the object to be sanitized without having to pass through a support material. The solid part of the support, such as, for example, the threads, are manufactured from materials, as described above, which allow some or all of the sanitizing radiation to pass through, so that the object to be sanitized can be essentially, completely exposed to the sanitizing radiation.

The support can be of any desirable shape such as, for example, a basket, a hammock, a flat bed, or other such form as desired.

The flexible support may be permanently attached inside the compartment of the apparatus or it may be removable wherein all, or a part of the object to be sanitized is place in the support and then attached inside the compartment. After sanitization, the support, and object, can be removed. This allows position of the object in the support as desired.

The apparatus may be constructed to allow the support to rotate in the compartment during sanitization to more efficiently and effectively expose the object to sanitizing radiation, such as, for example, allowing the “line-of-sight radiation to infringe into seams, holes, depressions that may normally be hidden form the radiation.

The sanitizing radiation is a line-of-sight process in which the radiation impinges directly onto the surfaces of objects. The line-of-light may be an unimpeded straight line directly from the source to the surface. The line-of-sight includes configurations wherein the sanitizing radiation is reflected from mirrors or other reflecting surfaces, which direct the sanitizing radiation onto surfaces which may not be in the direct line from the radiation source to the surface, thus allowing an increase in the amount of surface that can be sanitized during the sanitation process. As such, anything in the way of the direct line from the irradiation source to the surface, such as dirt particles and the like on the surface, will prevent those covered surface areas from being sanitized. This is a recognized issue and has been improved, in one case, by rotating the object to be sanitized so that more surface can be aligned in a line-of-line configuration with the radiation source. This problem has been further improved by providing mirrors and/or mirrored surfaces that reflect the sanitizing radiation at different angles to areas, again in a line-of-sight configuration, that would not be accessible in a straight line-of-sight configuration. The intensity of the sanitization is reduced the further it has to travel, in inverse ratio of the square of the distance, such that as the radiation gets reflected around the chamber the distance the radiation travels increases and the intensity of the radiation decreases. Thus any sanitization that depends on reflected sanitization radiation will require a longer radiation time to be effective as a sanitization device and/or process. Reflective radiation, in the current disclosure, is complimentary to the main line-of-sight radiation.

The current disclosure provides for a component for reducing debris that resides on the surface of the object to be sanitized so that, when in use, the radiation may reach those areas on the surface of the object to deactivate infectious organisms which would not have been exposed if the debris had not been removed. In some embodiments, the component for reducing debris may be positioned proximal to the ingress either after the ingress such that debris reducing component is inside the compartment, or before the ingress, such that the debris reducing component is outside the compartment. In either configuration the object to be sanitized is placed through the debris reducing component directly into the flexible support in the compartment prior to being exposed to sanitizing radiation. In other embodiments of the current disclosure the object to be sanitized may experience one or more of the debris removing components when placed into the flexible support in the compartment of the apparatus.

The component for reducing debris may be a brush mechanism wherein the mechanism may be positioned at the ingress, either in front of or behind the ingress, through which the object to be sanitized is placed. The brushes may be made of any of a number of suitable material, such as, for example, nylon, polyester, or other materials known in the art. As the object moves through the ingress the brush or brushes contact the surface of the object and remove a substantial amount of debris. In operation the object, which now has a reduced amount of debris on its surface, can be irradiated with sanitizing radiation more effectively than if the brushes were not present.

After sanitization the object may be removed from the component without passing through debris-reducing component or the object may pass back through the brush mechanism. In the former case the brushing mechanism may lift out of the way when a door, or cover, is lifted to allow access to the object. This may be done with an automatic clipping mechanism that engages when the door, or cover, is closed such that when the door, or cover, is opened the brushing mechanism stays removable attached to the door, or cover. When the device is removed and door or cover is replaced, the brushing mechanism is disengaged from the door, or cover, ready for the next object to be placed through it. In other embodiments, after sanitization, the object passes back through the brushing mechanism when being removed which may or may not redeposit some of the original debris. The removal of debris is primarily to allow the sanitizing radiation to impinge on the surface areas under the debris, although removal of debris serves other purposes such as cleanliness. In other embodiments the compartment may include an egress through which the now radiation sanitized object can pass through as it removed from the apparatus without going through the brush mechanism.

In other embodiments the component for removing debris may be one or more rollers having a surface onto which debris may be attracted, such as, for example, a tacky surface or an electrostatic surface. When the object to be sanitized is placed through the one or more rollers the debris gets picked up onto the surfaces of the roller thus exposing more surface area on the object to receive sanitizing radiation. The rollers may be removed, cleaned and replaced during routine maintenance to remove the accumulated debris that has been removed, or there may be a removal surface layer to which the debris has attached which can then be peeled off and replaced with a new layer. Additionally, there may be a number of layers of debris attaching material which, when the first, debris loaded layer is removed, the layer behind it to is now exposed and can be used to attach debris, such as, for example, a roll of adhesive tape with adhesive layer directed outwardly.

In other embodiments the component for removing debris may be an air impingement device wherein air is directed across one or more surfaces of the object to be sanitized. The air may impinge at the ingress and be applied as the object is being placed into the compartment. Alternatively the object may be placed into the compartment and air then is impinged onto one or more of the object's surfaces. At this stage the compartment may be open or closed.

The component for reducing debris may be a wiping mechanism wherein the component is attached to the compartment or apparatus and contains wipes. The wipes can be used by the user to physically wipe the surfaces of the object to be sanitized to reduce debris on the object's surface, prior to placing the object into the apparatus through the ingress for sanitization. The wipes may be comprised of, for example, cloth, paper, plastic or other material which can be used to wipe a surface. The wipes may be comprised of, for example, liquids, pastes and electrostatic capabilities which can aid in debris removal. The wipes further may be comprised of antibacterial ingredients which may aid in the sanitization of the object, including sodium hypochlorite and metal ions, such as, for example, silver, copper or zinc ions.

In further embodiments, the object to be sanitized may be a pass-through system in which the object is continuously conveyed through the compartment by such devices as a conveyor belt, or rollers which constitute the flexible support of the current disclosure. The debris removing component may be brushes, tacky rollers, air impingement and the like. Further examples of the debris removing embodiments of the current disclosure are presented in U.S. Pat. No. 9,265,849B2 to Kerr, herein incorporated by reference.

The apparatus and/or compartment are designed so that all or essentially all of the sanitizing radiation is prevented from escaping the compartment. Other, non-sanitizing radiation, such as visible light, may be allowed to escape such as, though a window through which the object to be sanitized can be seen. The window may be made of any material which blocks, or absorbs, UV radiation while allowing harmless visible to pass through.

The apparatus includes a radiometer sensor which senses the amount of radiation that the sanitizing bulbs have emitted. The sensor is electrically connected to a logic device which determines the required amount of sanitizing radiation to be emitted from the sanitizing bulbs. The logic device may be programmed for specific infectious organisms or programmed for general sanitization as desired, including, for example, time of sanitization, amount of energy, and the like. In the case of a conveying device, the logic device determines the speed with which the object is conveyed through the compartment. The logic device may be capable of reprogramming as desired.

The apparatus may contain a switching device with activates the sanitization process when the door or cover completely encloses the compartment so that essentially no radiation is allowed to emit from the apparatus. If the door or cover should prematurely open the switching device can turn the sanitization bulbs off.

The apparatus may also contain a signaling device which notifies the user when the sanitization process has completed and the object may be removed from the compartment. These devices include, for example, one or more lights, color-changing tabs, audio signals or combination.

The signaling component may also take the form of a window in the top or side of the apparatus through which only visible light from the radiation source can be seen when the radiation source is emitting radiation. The window is comprised is of material that is absorbent to harmful radiation such as UV radiation. In this manner a user can observe when the radiation source is emitting sanitizing radiation and when it has completed its operation and it is safe for the user to remove the object through an ingress, egress or other method.

While not to be restricted, the apparatus may contain any number of additional components, such as, for example, an anchoring mechanism for anchoring the apparatus to a table, a bench or other solid object to prevent unauthorized removal.

Objects suitable for sanitization using the apparatuses and methods disclosed herein may range from cell phones and TV remotes, to other handheld devices, smart pads, laptops, keypads, jewelry, keys, credit cards, money, toiletry items, food related items including food items that come into contact with food, such as trays, dental and medical equipment and devices, writing utensils, kitchen utensils, books, magazines, gaming items like dice and paying cards, toys, balls, persona hygiene items, such as combs, brushes, toothbrushes, baby related objects like rubber nipples, containers, and other objects which can carry infectious organisms from one person to another. The apparatus may be configured to enclose large objects such as shopping carts, bicycles, large machinery, arcade video games and other large objects which can carry infectious organisms from one person to another. Sports equipment, child related objects such as toys, cribs, playpens and the like, furniture are also suitable.

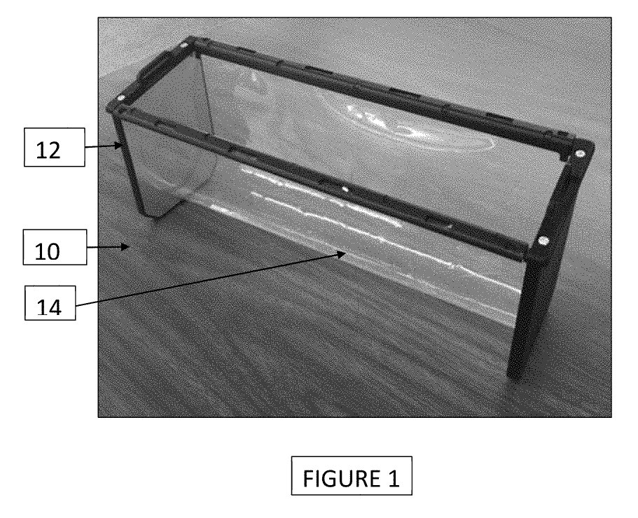

Drawing attention to FIG. 1 is a photo of one embodiment of the flexible support 10 of the current disclosure. The support is removable and comprises a frame 12 and a fluorinated polyethylene thin film support basket 14. In practice, the object to be sanitized, not shown, is placed into the basket 14 and the support 10 containing the object is placed into the sanitizing device.

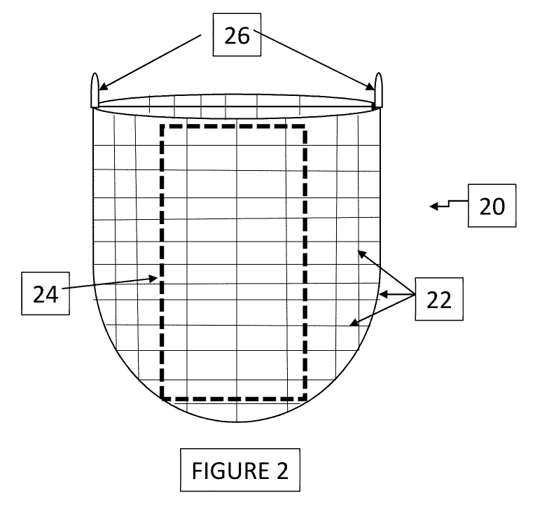

FIG. 2 is a graphic representation of another embodiment of the flexible support 20 of the current disclosure. Here is shown a mesh basket 20, made from perfluoropolyethylene fibers 22 into which the object to be sanitized 24 is placed. The basket has clips 26 that are used to removably attach and suspend the basket and object in the compartment of the device for sanitization.

I claim:

1. An apparatus for sanitizing an object, comprising:
a. a compartment comprising a top portion, a bottom portion and side portions and configured to enclose an object to be sanitized;
b. At least one radiation source within the compartment, wherein the at least one radiation source can provide UV radiation capable of substantially deactivating infectious organisms; and
c. A flexible support for holding and suspending the object to be sanitized within the compartment,
wherein the support comprises material capable of transmitting radiation capable of substantially deactivating infectious organisms.

2. The apparatus of claim 1, wherein the support comprises glass, a polymer or a combination.

3. The apparatus of claim 2, wherein the support comprises polymethylmethacrylate, fluoropolymers, fluorinated poly-co-ethylene-propylene, polythene, silicone, polymethylpentene, polyolefin, fluorinated polyolefin, cyclic olefin polymers, fluorinated cyclic polyolefin, polyethylene, perfluorinated polyethylene, polypropylene, perfluorinated polypropylene, or combination thereof.

4. The apparatus of claim 1, wherein the support is flexible and conformable around at least a portion of the object.

5. The apparatus of claim 1, wherein the support is a thin film, perforated sheet, a woven construction or a mesh.

6. The apparatus of claim 1, further comprising a rotating mechanism for rotating the support.

7. The apparatus of claim 1, wherein the UV radiation is between about 250 nm and about 280 nm.

8. The apparatus of claim 1, wherein the UV radiation is about 253.7 nm.

9. The apparatus of claim 1, further comprising a component for reducing the amount of debris on the surface of the object to be sanitized.

10. The apparatus of claim 1, further comprising electronic connectors for charging devices, connecting electronic devices, communication devices, internet devices, and combinations thereof.

11. An apparatus for sanitizing an object, comprising:
a. a compartment comprising a top portion, a bottom portion and side portions and configured to enclose an object to be sanitized;
b. At least one radiation source within the compartment, wherein the at least one radiation source can provide UV radiation capable of substantially deactivating infectious organisms; and
c. A removably attached flexible support for holding and suspending the object to be sanitized within the compartment,
wherein the support comprises material capable of transmitting radiation capable of substantially deactivating infectious organisms.

12. The apparatus of claim 11, wherein the support comprises glass, a polymer or a combination.

13. The apparatus of claim 12, wherein the support comprises polymethylmethacrylate, fluoropolymers, fluorinated ethylene propylene, polythene, silicone, polymethylpentene, cyclic olefin polymers, polyethylene or combination thereof.

14. The apparatus of claim 11, wherein the support is flexible and conformable around at least a portion of the object.

15. The apparatus of claim 11, wherein the support is a perforated sheet, a woven construction or a mesh.

16. The apparatus of claim 11, further comprising a rotating mechanism for rotating the support.

17. The apparatus of claim 11, wherein the UV radiation is between about 250 nm and about 280 nm.

18. The apparatus of claim 11, wherein the UV radiation is about 253.7 nm.

19. The apparatus of claim 11, further comprising a component for reducing the amount of debris on the surface of the object to be sanitized.
